(19) United States
(12) Patent Application Publication
(10) Pub. No.: US 2007/0086949 A1
(43) Pub. Date: Apr. 19, 2007

(54) Method of bioimaging using nanocrystals of fluorescent dyes

(76) Inventors: Paras N. Prasad, Williamsville, NY (US); Koichi Baba, Sendai (JP); Haridas Pudavar, N. Tonawanda, NY (US); Indrajit Roy, Amherst, NY (US); Tymish Ohulchanskyy, Kenmore, NY (US); Hachiro Nakanishi, Sendai (JP); Akito Masuhara, Sendai (JP); Hitoshi Kasai, Sendai (JP)

Correspondence Address: HODGSON RUSS LLP, ONE M & T PLAZA, SUITE 2000, BUFFALO, NY, 14203-2391, US

(21) Appl. No.: 11/471,390

(22) Filed: Jun. 20, 2006

Related U.S. Application Data
(60) Provisional application No. 60/692,145, filed on Jun. 20, 2005.

(51) Int. Cl. A61K 49/00; A61K 31/555; A61K 31/4745; A61K 31/409

(52) U.S. Cl. 424/9.6; 514/185; 514/410; 977/927

(57) ABSTRACT

This invention provides nanocrystals or polymer doped nanocrystals of hydrophobic organic fluorescent dyes as stable dispersions in an aqueous system. The dispersions can be prepared without stabilizers such as surfactants and the like. The aqueous dispersions of the nanocrystals or the polymer doped nanocrystals can be used for bioimaging.

This application claims priority to U.S. Provisional Application No. 60/692,145 filed on Jun. 20, 2005, the disclosure of which is incorporated herein by reference.

This invention was made with funds from United States Air Force/AFOSR Grant no. F49620-0101-0358. The Government has certain rights in the invention.

### FIELD OF THE INVENTION

This invention relates generally to the field of bioimaging and more particularly provides compositions comprising nanocrystals of fluorescent dyes and methods for using those in bioimaging.

### BACKGROUND OF THE INVENTION

Imaging of biological systems has gained considerable importance not only as a research tool, but also in the prevention and treatment of various diseased conditions. Imaging of cells and tissues requires the use of dyes which can enter cells and then be amenable to visualization.

Organic dyes which are potentially useful in bioimaging, such as infrared emitting dyes and two photon excitable fluorescent dyes, are generally hydrophobic and therefore poorly soluble in water, or even insoluble. Therefore preparation of aqueous dispersion of such dyes requires special formulation techniques. Surfactants are usually helpful for preparation of water dispersion of hydrophobic nanoparticles. However many surfactants themselves, or in some cases the byproducts produced during preparation of these surfactant based dye dispersions, tend to increase the cytotoxicity of the preparation. Therefore, the surfactant based methods for preparation of water dispersion of hydrophobic dye are not preferable for biological applications.

Because of hydrophobicity concerns, only a limited variety of dyes can be used for biological imaging. Currently used dyes include the rhodamine and coumarine family as well as their derivatives. Other attractive dyes, such as perylene and tetraphenyl-1,3-butadiene (TPB), which have high quantum yield, have been used in organic electro luminescence devices but not for biological imaging. Although such dyes have the potential to be useful for bioimaging, useful formulations comprising such dyes have not heretofore been developed.

An additional consideration in identifying useful bioimaging dyes is the size of the dye particles. Size is a critical parameter for in vitro and in vivo use. For example, for confocal fluorescence imaging of live cells, the size of fluorescent dye particles is preferably less than 100 nm for efficient particle uptake and reduced light scattering. For in vivo experiments, size is more critical and is preferably less than 50 nm to evade capture by reticuloendothelial system. It is believed that there is no current method which can produce dye compositions to satisfy or overcome the above conditions and problems.

### SUMMARY OF THE INVENTION

In the present invention, hydrophobic dyes having desirable optical properties ( e.g., fluorescence with high quantum yield in convenient spectral range allowing easy detection, easily available excitation wavelength etc.) are formulated as aqueous dispersed nanocrystals such that the dyes can be used for biological imaging of cells and tissues. Such imaging can be used as part of a diagnostic or therapeutic approach in the treatment or prevention of various diseased conditions.

In one embodiment, nanocrystals of the hydrophobic dye are generated by a simple two step method comprising the steps of dissolving millimolar amounts of a dye in an organic solvent to form a solution and then mixing a small amount of the organic solution with water to form the dye nanocrystals

In one variation of the above method, polymer doped dye nanocrystals are generated which exhibit reduced initial quenching of fluorescence in a comparison to the undoped nanocrystals. For preparation of polymer doped dye nanocrystals, the dye and polymer are dissolved in the organic solvent and the solution is then mixed with water to form the polymer doped nanocrystals.

For imaging of cells using the dye nanocrystals or polymer doped nanocrystals, the cells are contacted with the nanocrystals (such as by incubation or administration) for a time sufficient for penetration of the dye into the cells. Microinjection of the dye nanocrystals into the cells also can be used for cell imaging. The cells can then be visualized or imaged using standard methods.

### BRIEF DESCRIPTION OF THE FIGURES

FIG. 1: Structure of 1,1,4,4-Tetraphenyl-1,3-butadiene (TPB) and perylene.

FIG. 2: Absorption spectra (A) and fluorescent spectra (B) of perylene nanocrystal water dispersion.

FIG. 3: Absorption spectra (A) and fluorescent spectra (B) of TPB nanocrystal water dispersion.

FIG. 4: Laser scanning microscopy imaging of HeLa cells incubated with TPB nanocrystal water dispersion. Fluorescence (A) and light transmission (B) images are shown. TPB, 1 hour staining: 390 nm excitation.

FIG. 5: Laser scanning microscopy imaging of HeLa cells incubated with Perylene nanocrystal water dispersion. Fluorescence (A) and light transmission (B) images are shown. Perylene, 1 hour staining: 457 nm excitation.

FIG. 6: The control of size by controlling the dye concentration in perylene gnanocrystal preparation

FIG. 7: Chemical structures of (A) D1 and (B) BT101.

FIG. 8: A) Absorption and (B) Fluorescence spectra of D1 nanocrystals and PLGA doped D1 nanocrystals in water dispersion Excitation wavelength for fluorescence measurement was 532 nm.

FIG. 9: SEM images; (a) BT101 nanocrystals. (b) PLGA doped BT101 nanocrystals. The initial concentration of the polymer in acetone solution was (b) 0.8 g/ml The particle sizes are (a) 35±9 nm and (b) 36±8 nm, Scale bar indicates 200 nm.

FIG. 10: Fluorescence spectra of (A) one-photon and (B) two-photon excitation. The excitation wavelength was 377 nm for (A) and 800 nm for (B).

FIG. 11: Two-photon laser scanning microscopic imaging of KB cells incubated with BT101. KB cells were treated with BT101 nanocrystals Light transmission (a) and fluorescence (b) images are shown.

### DETAILED DESCRIPTION OF THE INVENTION

The present invention provides a method of imaging cells by using aqueous dispersions of hydrophobic dye molecules. The aqueous dispersions are prepared by dissolving the organic hydrophobic dye molecules in a water miscible organic solvent and adding the resultant solution to water resulting in the formation of nanocrystals. In a variation of this method, polymer doped dye nanocrystals can be used.

In the method of the present invention, nanocrystals and polymer doped nanocrystals are produced from suitable bioluminescent materials.

The term “Nanocrystal or “Nanocrytals” as used herein means a crystallite or crystallites of size ranging from 1 nm to 1000 nm. The term “polymer doped nanocrystal” as used herein means the nanocrystals as described above comprising one or more types of polymer molecules interspersed amongst the dye molecules. The percentage of polymer in a nanocrystal is less than 20% or 10%. Preferably, the polymer is less than 10%. The method of the present invention comprises contacting cells with the nanocrystal or polymer doped nanocrystal and imaging the cells.

The aqueous dispersion of the dye nanocrystals or polymer doped nanocrystals is prepared by dissolving the dye in an organic solvent and adding the dye- organic solvent solution to water resulting in the formation of nanocrystals. It has been shown by X-ray diffraction measurements that such structures are crystalline in nature (Kasai et al., 2000, 5:433-473). In one embodiment, a small amount of polymer and the organic dye are added to the organic solvent and then the polymer and dye containing organic solvent is added to water to result in the formation of polymer doped nanocrystals.

This method can be used for wide range of hydrophobic organic fluorophores (e.g., such as porphyrines, phtalocyanines, other heterocyclic low molecular compounds displaying fluorescence in organic solvent).

The present method for the preparation of hydrophobic organic dyes nanocrystals or polymer doped nanocrystals as water dispersions involves reprecipitation. The dyes which can be used in the present invention are those which are soluble in polar organic solvents but are insoluble in water. Non-limiting examples of such dyes are perylene, phthalocyanine, cyanine dyes, naphthalene and the like. Some key features of this method are
(i) Fabrication of hydrophobic organic nanocrystals or polymer doped nanocrystals as aqueous dispersion.
(ii) Surfactant free fabrication of hydrophobic organic nanocrystals or polymer doped nanocrystals aqueous dispersion.
(iii) No chemical reaction required for the preparation of hydrophobic organic nanocrystal or polymer doped nanocrystal aqueous dispersion.
(iv) No substantial fluorescence quenching for polymer doped nanocrystals for dyes which typically loses fluorescence in water environment.
(v) Control of particle size in the nanometer range.

In the present invention, the dye is dissolved in an organic solvent which is water miscible. Non-limiting examples of water miscible organic solvents include, but are not limited to, acetone, ethanol and dimethyl sulfoxide (DMSO), dimethylformamide (DHF), tetrahydrofuran (THF), ethyl acetate,methyl iso-butyl ketone, methyl acetate, methyl propyl ketone, iso-pentyl alcohol, iso-propyl alcohol, methyl alcohol, ethylene glycol monobutyl ether and propylene glycol monomethyl ether. Solvents which are not miscible with water i.e., hexane, chloroform and benzene are not suitable.

For example, the hydrophobic dye is dissolved in an organic solvent selected based on the drug solubility as described above, at millimolar concentration (ranging from 1 mM-100 mM based on dye solubility in the selected organic solvent). After preparation of the solution of the objective dye, microliter quantities of this solution is added to water.

To get a homogenous population of nanocrystals, it is preferable to add the dye-solvent solution quickly into water with continuous stirring. For example, the dye-solvent solution can be added to water using a micro-syringe while the water is stirred (such as magnetically at about 1000 rpm). After this two-step procedure, an aqueous dispersion of nanocrystals is obtained. While not intending to be bound by any particular theory, it is believed that as the surface of the nanocrystals is negatively charged (as determined by zeta potetntial measurements of few tens of mV), the dispersion is electrostatically stabilized to form water dispersions without aggregation.

The solvent can be removed from the preparation by dialysis after fabrication of nanocrystals. Dialysis can be carried out by routine methods, using a dialysis membrane of cut-off size 3.5 kD and above.

It has been observed that the nanocrystal composition as an aqueous dispersion exhibits little fluorescence Similar fluorescence quenching in dye aggregates is well known in case of molecular aggregates (Yuzhakov, 1992). However, when this formulation enters a cell, surprisingly, it was observed that the fluorescence ability is regained—likely by association of the nanocrystals with the cellular components such as lipid and/or protein molecules—and this allows for imaging of cells.

Quenching of fluorescence can be a concern. The reason for the fluorescence quenching of hydrophobic organic dyes in water, in many cases is because of intermolecular interaction between dye molecules due to the formation of molecular aggregates. Therefore, in one embodiment, if one needs to have detectable fluorescence at the initial stage, the dye can be mixed with a small amount of polymers to form polymer doped dye nanocrystals in which the polymer molecules are interspersed with the dye molecules. Another interest in these polymer doped nanocrystals comes from the potential to control the rigidity of the prepared nanocrystals. The incorporation of polymers can be optimized for controlled release of the dye molecules from the crystalline aggregated state.

The size of the nanocrystals or polymer doped nanocrystals can be varied by controlling various factors. For example the concentration of the dye in the organic solvent is an important factor. Size of the nanocrystals increases with increase in dye concentration and it can be adjusted from few tens of nanometers to few hundreds of nanometers, by tuning the concentration. Further, it is preferable to use water at a temperature such that a uniform distribution of the nanocrystals is obtained and there is no aggregation of the dye nanocrystals. Another factor is the ratio of the organic solvent to water. The volume of the organic solvent added to water is preferably about 10-100 times less than the volume of the water. For example, the volume of DMSO solution (containing the dye) is preferably about 200 μl in about 10 ml of water.

Thus, for each compound, the size of the nanocrytals can be easily controlled on nanoscale range (less than 100 nm) and, the fabricated nanocrystals are well dispersible in water. Generally nanocrystals of less than 150 nm, preferably less than 120 nm, and more preferably less than 100 nm. In one embodiment, the size is less than 50 nm. are useful for bioimaging. Since this is surfactant free technique requiring no chemical reactions, the resulting water dispersion of nanocrystals is safer than the conventional nanoparticle or water dispersion of these organic dyes used in biological applications. Examples of polymers that can be used include but not limited to Polylactides (PLA),Polyglycolides (PGA),Poly(lactide-co-glycolides) (PLGA), all possible co-polymer ratios, Polyanhydrides Polyorthoesters, and Polysiloxylanes can be used.
The present method satisfies the following criteria which are desirable for compositions for bioimaging:

(i) Surfactant free compositions (ii) No chemical reaction in fabrication of nanocrystals, (no possible chemical modification of the dyes or no additional component in the final preparation) (iii) Stable aqueous dispersion of nanocrystals, showing no precipitation during storage over a period of two months at room temperature (iv) Reduction in quenching of fluorescence for polymer doped nanocrystals and (v) Ability to control size of nanocrystals.

For using the compositions of the present invention in bioimaging, the composition is contacted with cells which are to be imaged. For example, cells can be incubated with the composition. Upon incubation, the nanocrystals enter the cells and the cells can then be visualized for imaging.

Any suitable method may be used to detect fluorescent dye delivered with nanocrystals in a target cell. In one embodiment, microscopy is used to detect the fluorescently labeled cell. Labeled cells can be detected using a fluorescence microscope, a multiphoton microscope, a confocal microscope, epifluorescence, two photon or any other in vitro or in vivo imaging method. Additionally computer aided analysis can be used to image or quantitate the fluorescence. An imprint of the cells' fluorescence can be recoded digitally. By this method, the morphology of the cells can be studied.

The present method can be used to diagnose a disease, disorder or condition. The target cells can be visualized in culture, but can also be visualized in vivo. Therefore, it is not necessary that the cells be removed from the body. Animal cells or tissues may be labeled and imaged in situ. The target cells or tissue may be contacted with the nanocrystals and imaged without disrupting the surrounding tissue or cells. A disease, disorder or condition that is characterized by a change in cell morphology, cell permeability or other parameter that can be detected by staining may be diagnosed according to the present method. For imaging cells in vivo, the composition can be administered by any standard method.

Examples of cells that can be studied by the method of the present invention include, but are not limited to, prokaryotic cells, eukaryotic cells, mammalian cells, and plant cells. Examples of mammalian cells include fibroblasts, epithelial cells, neural cells, intestinal cells, embryonic and adult stem cells, ovarian cells, liver cells, prostate cells, kidney cells, bladder cells, blood cells, yeast cells, bacteria cells, and immune cells. Additionally, cell lines including immortalized cells, can also be studied. Such cells include but are not limited to: HeLa, KB, UC1-107, MCF-7, Mia-Paca, Pac-1,TE-671 etc. Attractive dyes such as of infrared emitting dyes, two photon excitable dyes and dyes used for electroluminescent devices (perylene and TPB) are used for demonstration of this approach and are applied for biological imaging.

### EXAMPLE 1

To illustrate the invention, water dispersions of two hydrophobic dyes, 1,1,4,4-Tetraphenyl-1,3-butadiene (TPB) and Perylene were prepared and used for bioimaging as described below.

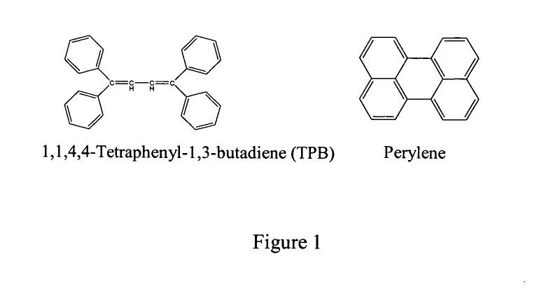

1,1,4,4-Tetraphenyl-1,3-butadiene (TPB) and Perylene (FIG. 1) are hydrophobic compounds and are therefore difficult to fabricate as nanoparticles in aqueous dispersion. However, we have successfully used this preparation method to produce TPB and perylene nanocrystals water dispersion.

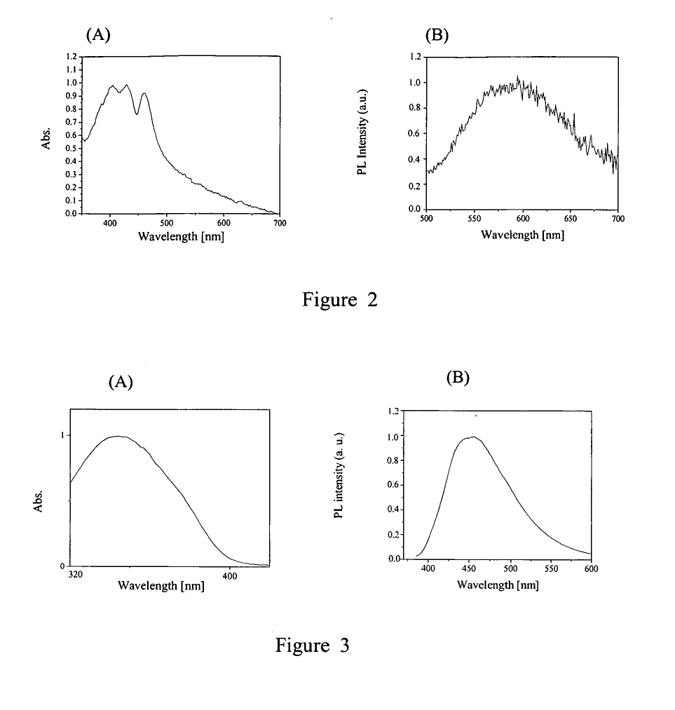

Briefly, 200 μl of dye acetone solution (10 mM concentration ) was injected into magnetically stirring water (10 ml). The average size of nanoparticle was estimated to vary from 50 nm to 200 nm depending on the concentration of original organic solution and water temperature. A homogenous dispersion of the dye nanocrystals without any noticeable aggregation or clumps was produced. Optical properties of the prepared nanocrystals were studied using UV-Visible and fluorescence spectroscopy. UV-visible absorption spectra as well as the fluorescence spectra of the nanocrystals were acquired using a Shimadzu 3600 UV spectrometer and a Jobin-Yvon Spectramax fluorimeter. Changes in absorption spectra as well as fluorescence intensity changes (such as quenching) were studied by comparing the spectra of nanocrystals in water with that of organic solution. The absorption and fluorescence spectra of nanocrystal water dispersions are shown in FIG. 2 and FIG. 3.

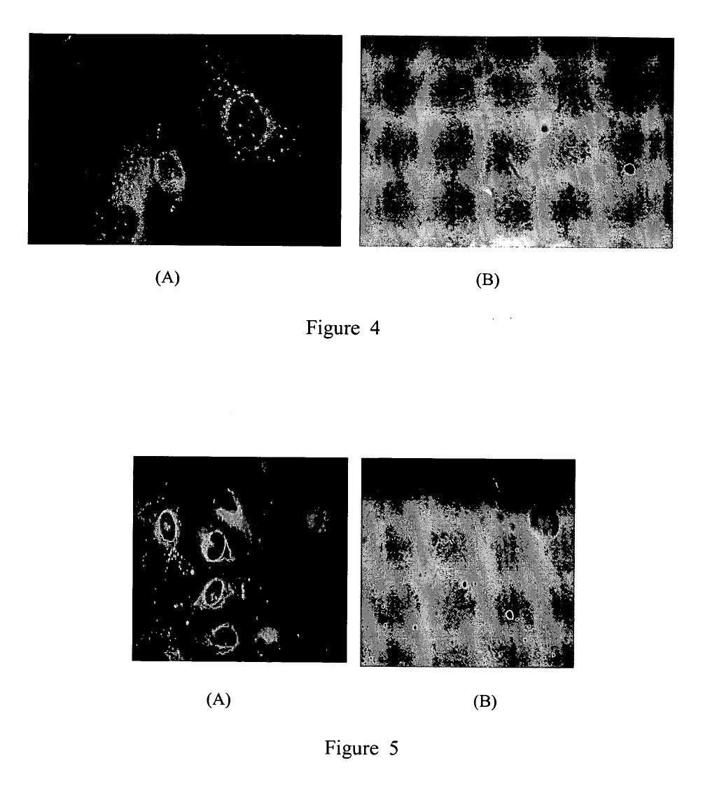

For cellular imaging, human cervix carcinoma cell line Hela was incubated with the prepared dye dispersion. The cell culture and imaging conditions are described in detail here. Human cervical carcinoma cell line (HeLa) was maintained in Dulbecco's modified eagle medium with 10% FBS according to the manufacturers instructions (American Type Culture Collection, Manassas, Va.). To study the uptake and imaging of HPPH nanocrystals, the cells were trypsinized and resuspended in the corresponding suitable media at a concentration of around 7.5×10⁵/ml. 60 μl of this suspension was transferred to each 35 mm culture plate and 2 ml of the corresponding full medium was added. These plates were then placed in an incubator at 37° C. with 5% CO₂ (VWR Scientific, model 2400). After 36 hours of incubation, the cells (about 60% confluency) were rinsed with PBS, and 2 ml of the corresponding fresh media was added to the plates. Finally, 50 μl of dye nanocrystals was added and mixed properly. Plates were returned to the incubator (37° C., 5% CO₂) for the required incubation period. 50 μl of dye/Tween80 micelles of same dye concentration was used as control and separate culture plates were treated and incubated for the same time period as in the case of nanocrystals. After each specific time interval of incubation, the plates were taken out, rinsed several times with sterile PBS and 2 ml of fresh serum free medium was added. The plates were incubated for another 10 minutes at 37° C. and were directly imaged under confocal a microscope. While not intending to be bound by any particular theory, it is believed that the homogeneous dispersion of these dyes in water enabled the dye to penetrate through cell-membrane and stain intracellular organelles within a short time (less than 1 hour). In case of perylene, dye seemed to be staining the entire cytoplasm with higher dye accumulation in vescicular bodies such as lysosomes. Obtained confocal images are shown in FIG. 4 and FIG. 5

### EXAMPLE 2

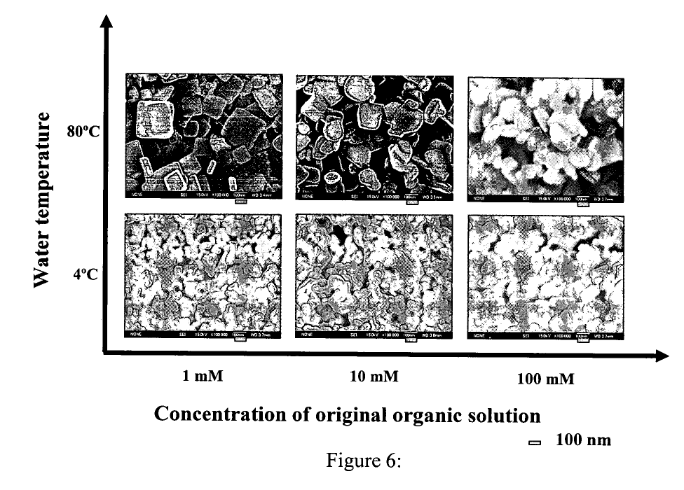

This example demonstrates the variation of nanocrystal size with concentration of the dye. For this experiment, perylene was dissolved on DMSO at 1 mM, 10 mM or 100 mM. The nanocrystals were prepared as described in Example 1 by adding to water at 4° C. or 80° C. As shown in FIG. 6, the size of the nanocrystals was observed to increase with increasing concentration of perylene. The size of the nanocrystals was also greater at the higher water temperature. Additionally, at the higher water temperature, the nanocrystals size distribution showed certain amount of polydispersion.

### EXAMPLE 3

This example describes the preparation of polymer doped nanocrystals. An infrared emitting dye (D1 ) and a two photon excitable fluorescent dye (BT101) were used as illustrative dyes.

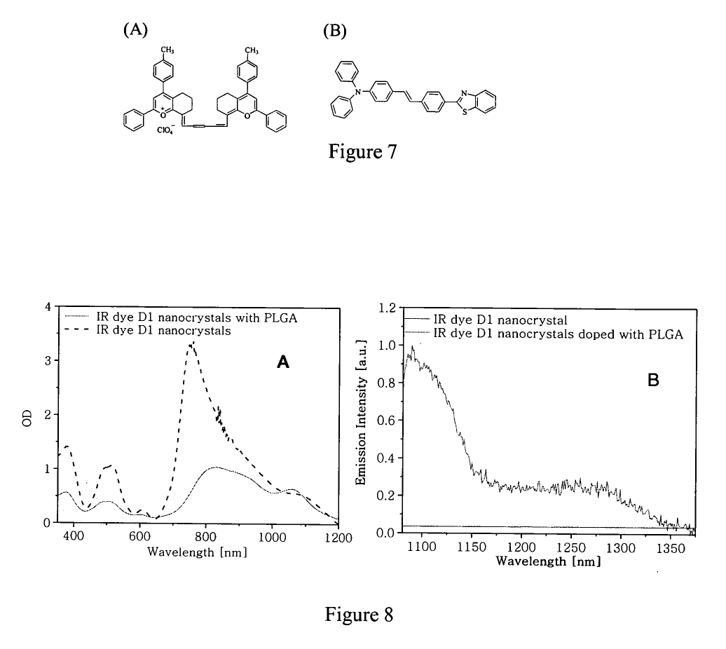

The infrared emitting dye D1 (FIG. 7A), shows fluorescence around 1.1 to 1.35 μm in organic solvents, but is significantly quenched in aqueous media. Two photon excitable fluorescent dye BT101 (FIG. 7B), has a polar D-π-A structure, in which the π-system is end-capped by an electron donor (D) and an electron acceptor (A)[2]. This structure is one of the most effective molecular models for both second- and third-order nonlinear optical materials, thus BT101 has great potential to be suitable for two-photon imaging. However, as with D1, BT101 lacks the necessary solubility properties to have application for biological systems.

The two-photon excitable fluorescent dye (BT101) was synthesized as reported by Lin et al. [2] . . . Poly (D,L-lactide-co-glycolide) (50:50) (PLGA) and polystyrene (Mw. 800) were purchased from Aldrich (St. Louis, Mo.). The KB cell line was obtained from the American Type Tissue Culture collection (ATCC, Rockville, Md.).

D1 and BT101 were dissolved into DMSO and acetone at concentrations of 1.3 mM and 0.2 mM, respectively. D1 or BT101 nanocrystal water dispersion were prepared by injecting the dye solution (200 μl) into 10 ml of magnetically stirred water (1100 rpm on magnetic stirrer) at room temperature.

The preparation of PLGA doped nanocrystal water dispersion was as follows. First mixed solution of D1 and PLGA DMSO solution was prepared by simultaneously dissolving both components in DMSO(the concentration was 1.3 mM for D1, 0.8 mg/ml for PLGA). 200 μl of this solution was injected into 10 ml of magnetically stirred water at room temperature.

The D1 nanocrystal dye shows quenched emission in aqueous media Upon incorporation of polymer into the nanocrystal, the fluorescence quenching is expected to decrease and the fluorescence yield is expected to increase. This is based on observations of a similar phenomenon when dye aggregation and, correspondingly, fluorescence quenching are reduced when dye is interdispersed together with a another compound, which molecules prevent aggregation of dye molecules causing fluorescence lost. FIG. 8a and 8b shows the absorption and emission spectra of D1 nanocrystals as well as PLGA doped D1 nanocrystals. The size and shape of nanoparticles was determined by scanning electron microscopy (SEM) (S4000; Hitachi). The size distribution was calculated by manually counting a large number of particles in the SEM picture. The size distribution was expressed as means ± standard deviation. Optical properties of D1/DMSO solution, D1/water dispersion and BT101/water dispersion were characterized by using a UV-3101 PC Spectrophotometer (Shimadzu; UV absorption spectra) and a Fluorolog-3 Spectrofluorometer (Jobin Yvon; fluorescence spectra).

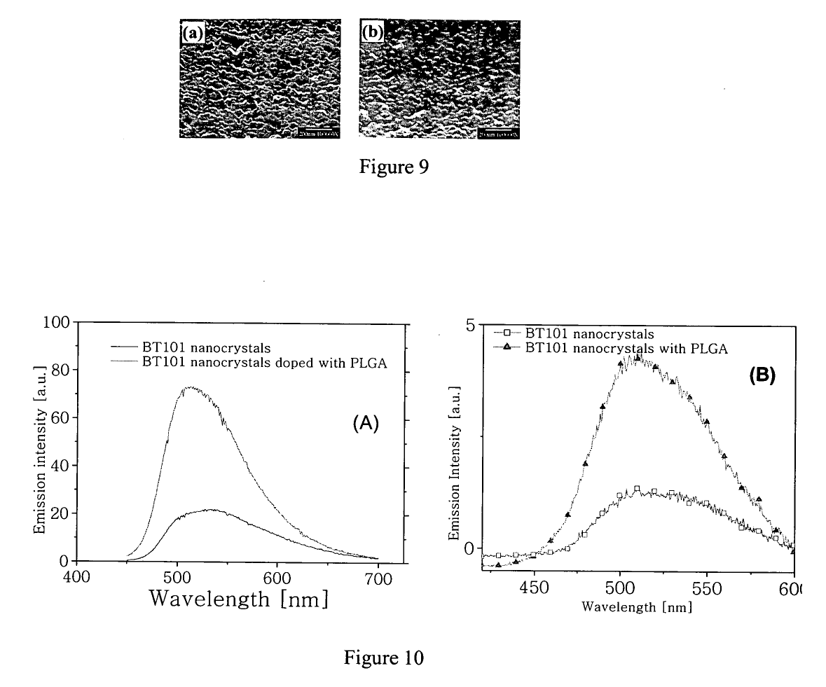

The size of BT101 nanocrystals was 35+9 nm. A SEM image of this preparation is shown in FIG. 9a. An image of polymer doped nanocrystals is shown in FIG. 9b where the size of of the nanocrystals was 36±8 nm. By using the method described herein we have successfully fabricated PLGA doped nanocrystals which are smaller than 100 nm. The BT101 nanocrystals also showed significant fluorescence quenching in water (FIG. 10). But with the addition of small amount of PLGA polymer, the initial fluorescence improved significantly. A point to be noted is that on cellular uptake, both pure nanocrystals and polymer doped nanocrystals recovered fluorescence to their full extent.

Uptake of nanocrystal preparation or polymer doped nanocrystal preparation was performed in the oral adenocarcinoma cell line, KB. Cell were grown and maintained in Delbecco's minimal essential media (DMEM) supplemented with 10% fetal calf serum. For uptake and imaging, cells were trypsinized, resuspended and replated in 60 mm culture dishes at a concentration of 7.5×104 cells/ml. The plates were incubated overnight under 37° C. in 5% of CO₂. Cell monolayers were rinsed with phosphate buffered saline, pH 7.2 (PBS) and 150 μl of aqueous dispersion of BT101 nanocrystals were added to 5 ml cell plate. The final concentration of BT101 in each plate was 0.12 M. After 24 h of incubation, the plates were rinsed with PBS and 5 ml of DMEM added to each plate. Cells were directly imaged using a confocal laser scanning system (MRC-1024, Bio-Rad, Richmond, Calif.). Two-photon imaging was performed using Ti-sapphire laser (Tsunami, Spectra Physics), pumped by a diode-pumped laser (Millenia, Spectra Physics), providing femtosecond pulses at 800 nm for excitation.

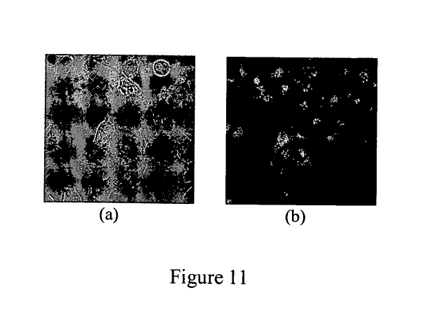

The results of cell imaging are shown in FIG. 11 a-b. Confocal laser microscopy imaging revealed fluorescent staining pattern of cells incubated with BT101 nanocrystals.

While this invention has been described through specific embodiments described herein, those skilled in the art can make routine modifications to the embodiments, which modifications are intended to be within the scope of the invention.

### REFERENCES

1. P. Rolfe, Annu. Rev. Biomed. Eng. 2, 715 (2000).
2. T.-C. Lin, G. S. He, P. N. Prasad and L. -S. Tan, J Mater. Chem. 14, 982 (2004).
3. D. Horn and J. Rieger, Angew. Chem. Int. Ed. 40, 4330 (2001).
4. R. A. Jain, Biomaterials 21, 2475 (2000).
5. H. Kasai, H. S. Nalwa, H. Oikawa, S. Okada, H. Oikawa, H. Matsuda, N. Minami, A. Kakuta, K. Ono, A. Mukoh and H. Nakanishi, Jpn. J Appl. Phys. 31, L1132 (1992).
6. H. Kasai, H. S. Nalwa, S. Okada, H. Oikawa and H. Nakanishi, in Handbook of Nanostructure Materials and Nanotechnology, edited by H. S. Nalwa (Academic Press, San Diego, 2000), Vol. 5, p. 433.
7. A. Sinicropi, W. M. Nau and M. Olivuccl,
Photochem. Photobiol. Sci. 1, 537 (2002).
8. U. P. Andley, J. N. Liang and B. Chakrabarti, Biochemistry 21, 1853 (1982).
9. R. F. Khairutdinov and N. Serpone, J Phys. Chem. B 103, 761 (1999).
10. G. B. Behera, B. K. Mishra, P. K. Behera and M. Panda, Adv. Colloid. Interface. Sci. 82, 1 (1999).
11. Yuzhakov, V. I., Aggregation of Dye Molecules and Its Effect on Spectral-Luminescent Properties of Solutions. Uspekhi Khimii 1992, 61, (6), 1114-1141.

1. A method for imaging of cells comprising the steps of contacting the cells with an aqueous dispersion comprising nanocrystals of a hydrophobic organic dye, and upon penetration of the dispersion into the cells, obtaining an image of the cells.

2. The method of claim 1 wherein the hydrophobic organic dye is selected from the group consisting of porphyrines, phthalocyanine, cyanine and naphthalene.

3. The method of claim 1, wherein the hydrophobic organic dye is selected from the group consisting of 1,1,4,4-tetraphenyl-1,3-butadiene (TPB), perylene, D1 as shown in FIG. 7A and BT101 as shown in FIG. 7B.

4. The method of claim 1, wherein an image of the cells is obtained by using confocal microscopy.

5. The method of claim 1, wherein the aqueous dispersion of nanocrystals is prepared by dissolving the hydrophobic organic dye in a water miscible solvent to form a solution and adding the said solution into water.

6. The method of claim 5, wherein the ratio of water miscible solvent to water is about 1:10 to 1:100.

7. The method of claim 1, wherein the aqueous dispersion is substantially free of surfactants.

8. The method of claim 5, wherein the water miscible solvent is selected from the group consisting of dimethyl sulfoxide (DMSO), dimethylformamide (DHF), tetrahydrofuran (THF), ethyl acetate, methyl iso-butyl ketone, methyl acetate, methyl propyl ketone, iso-pentyl alcohol, iso-propyl alcohol, methyl alcohol, ethylene glycol monobutyl ether and propylene glycol monomethyl ether.

9. The method of claim 1, wherein the mean size of the nanocrystals is less than 150 nm.

10. The method of claim 9, wherein the mean size of the nanocrystals is less than 100 nm.

11. The method of claim 11, wherein the mean size of the nanocrystals is between 30 to 50 nm.

12. The method of claim 5 wherein a polymer is also dissolved in the water miscible solvent such that the concentration of the polymer in the nanocrystals is less than 20%.

13. The method of claim 12, wherein the concentration of the polymer is less than 10%.

14. The method of claim 14, wherein the polymer is selected from the group consisting of Polylactides (PLA), Polyglycolides (PGA), Poly(lactide-co-glycolides) (PLGA), co-polymers thereof, Polyanhydrides, Polyorthoesters and Polysiloxylanes.

15. The method of claim 14, wherein the polymer is PLGA.

16. The method of claim 1, wherein the cells are imaged in vitro.

17. The method of claim 1, wherein the cells are imaged in vivo.

18. A composition comprising nanocrystals of a hydrophobic organic dye selected from the group consisting of porphyrines, phthalocyanine, cyanine and naphthalene.

19. The composition of claim 18, wherein the dye is selected from the group consisting of 1,1,4,4-tetraphenyl-1,3-butadiene (TPB), perylene, D1 as shown in FIG. 7A and BT101 as shown in FIG. 7B.

20. The composition of claim 19, wherein the nanocrystals further comprise a polymer such that the polymer is less than 20 wt %.
